(19) United States
(12) Patent Application Publication
(10) Pub. No.: US 2006/0052710 A1
(43) Pub. Date: Mar. 9, 2006

(54) Endoscope apparatus and fluorescence detection method using endoscope apparatus

(75) Inventors: Naoki Miura, Tokyo (JP); Toshiaki Watanabe, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, TOKYO (JP)

(21) Appl. No.: 11/219,195

(22) Filed: Sep. 2, 2005

(30) Foreign Application Priority Data
Sep. 8, 2004 (JP) 2004-260617
Jul. 22, 2005 (JP) 2005-212229

(51) Int. Cl. A61B 6/00

(52) U.S. Cl. 600/476

(57) ABSTRACT

An endoscope apparatus includes an illumination unit configured to emit illumination light including light in a visible spectral band and excitation light that generates fluorescence in a plurality of different infrared bands toward an examination region in a body to be examined, an imaging unit configured to capture the illumination light reflected at the examination region and the fluorescence generated at the examination region and disposed at a distal end of an insertion section to be inserted inside the body to be examined, and a cut filter configured to block at least the excitation light in a plurality of spectral bands, the cut filter being disposed adjacent to the imaging unit on a light-incident side.

### BACKGROUND OF THE INVENTION

1. Field of the Invention

The present invention relates to an endoscope apparatus and to a method for detecting fluorescence using an endoscope apparatus. This application is based on Japanese Patent Application Nos. 2004-260617 and 2005-212229, the contents of which are incorporated herein by reference.

2. Description of Related Art

Technologies for examining biological tissue for degeneration or disease, such as cancer, by detecting autofluorescence from a living body or fluorescence generated by a chemical agent administered to a living body have hitherto been known.

For example, when excitation light is irradiated onto biological tissue, fluorescence having a longer wavelength than the excitation light is generated. Recently, it has become apparent that a living body includes materials that emit fluorescence when irradiated with excitation light and that such materials and disease are interrelated. There are also known fluorescent agents that emit fluorescence when degraded by enzymes present in a living cell. Since cancer cells secrete a larger amount of such enzyme compared to normal cells, by injecting this fluorescent agent into a body, a cancerous part can be diagnosed, and the amount of enzyme secreted can be measured.

A method for observing such fluorescence by an endoscope has been proposed. By using an endoscope in such a method, diagnosis of cancer in a body cavity is possible.

Endoscopes used in such diagnosis may be a fiber scope or a video scope. A fiber scope guides light through an optical fiber to outside the body, and a video scope, for observing an image formed by the light, in which a charge-coupled device (CCD) is provided at the distal end of the endoscope apparatus. Either type of endoscope may be used to examine diseases such as cancer in a body cavity. A fiber scope is advantageous in that its diameter can be made smaller than a video scope, whereas a video scope is advantageous in that the resolution of the obtained image is higher than that of a fiber scope.

Various technologies have been proposed for video scopes that are suitable for the above-described diagnosis because of their reduced-diameter and capability of observing light in two different spectral bands.

Japanese Unexamined Patent Application Publication No. HEI-8-140928 describes an endoscope including one CCD for detecting visible light and another CCD for detecting fluorescence of a predetermined wavelength.

Such an endoscope configured to detect fluorescence of a predetermined wavelength, for example, is not capable of detecting the extent to which sites a fluorescent agent that generates fluorescence in cancer cells has spread after the fluorescent agent is administered to a patient. Therefore, when fluorescence is not detected, it is impossible to determine whether this is because cancer cells are not present or because the fluorescent agent has not spread throughout the site. For this reason, it has been difficult to apply this technology to examining cancer cells.

Japanese Unexamined Patent Application Publication No. HEI-8-224208 discloses an endoscope including a filter capable of transmitting light in at least two different spectral bands and a CCD sensitive to a plurality of spectral bands, including the two different spectral bands of light that pass through the filter.

For example, if fluorescence in at least two different spectral bands can be detected, the problem of Japanese Unexamined Patent Application Publication No. HEI-8-140928 can be solved by using a fluorescent agent that generates fluorescence of a predetermined wavelength when injected into cancer cells and another fluorescent agent that generates fluorescence of a wavelength different from the predetermined wavelength. In other words, by using two different fluorescent agents, the spread of the fluorescent agents throughout a site can be detected, and, as a result, cancer cells can be easily examined since, when fluorescence is not detected, it can be determined whether this is because cancer cells do not exist or because the fluorescent agents have not spread throughout the site.

However, Japanese Unexamined Patent Application Publication No. HEI-8-224208 has a disadvantage in that, in order to simultaneously observe visible light and fluorescence, at least two image pickup devices are required, namely, one image pickup device for observing visible light and another image pickup device for observing fluorescence.

### BRIEF SUMMARY OF THE INVENTION

The present invention has been conceived in light of these circumstances, and it is an object of the present invention to provide an endoscope apparatus capable of easily observing an examination region and a fluorescence detection method using an endoscope apparatus.

The present invention provides the following solution.

A first aspect of the present invention includes an illumination unit configured to emit illumination light including light in a visible spectral band and excitation light in a plurality of spectral bands that generates fluorescence in a plurality of different infrared bands toward an examination region in a body to be examined, an imaging unit configured to capture the illumination light reflected at the examination region and the fluorescence generated at the examination region and disposed at a distal end of an insertion section to be inserted inside the body to be examined, and a cut filter configured to block at least the excitation light in the plurality of spectral bands, the cut filter being disposed adjacent to the imaging unit on a light-incident side.

According to the first aspect of the present invention, the endoscope apparatus is capable of observing light reflected from the examination region by the imaging unit. Therefore, the region being examined with the endoscope apparatus can be easily determined. Accordingly, the region can be easily located, and it can be easily determined whether the region is the examination region.

For example, when using a fluorescent agent that is degraded by an enzyme present in predetermined cells in the examination region and generates fluorescence of a predetermined wavelength due to excitation light and another fluorescent agent that generates fluorescence of a wavelength different from the predetermined wavelength due to excitation light, the imaging unit can detect the area in which the fluorescent agents have spread throughout. Consequently, when fluorescence is not detected, it can be determined whether this is because the predetermined cells are not present in the examination region or because the fluorescent agent has not spread throughout examination region. As a result, an examination of the examination region can be carried out easily.

For example, when using a fluorescent agent that is degraded by an enzyme present in predetermined cells in the examination region and generates fluorescence of a predetermined wavelength due to excitation light and another fluorescent agent that is degraded by an enzyme present in other cells and generates fluorescence of a predetermined wavelength by excitation light, a plurality of predetermined cells can be observed simultaneously. Since a plurality of predetermined cells can be examined by observing the examination region once, observation of the examination region can be easily carried out.

According to a second aspect of the present invention, an endoscope apparatus includes an illumination unit configured to emit illumination light including light in a visible spectral band and excitation light in a plurality of spectral bands that generates fluorescence in a plurality of different infrared bands toward an examination region in a body to be examined, a first imaging unit configured to capture at least a portion of the fluorescence generated at the examination region and disposed at a distal end of an insertion section to be inserted in the body to be examined, a second imaging unit configured to capture all or a portion of the fluorescence, which is fluorescence other than the fluorescence captured by the first image pickup and included in at least the illumination light reflected at the examination region and the fluorescence generated at the examination region, and disposed at the distal end of the insertion section to be inserted in the body to be examined, a first cut filter configured to blocks at least excitation light that generates the aforesaid portion of the fluorescence and disposed adjacent to the first imaging unit on a light-incident side, and a second cut filter configured to block all or a portion of excitation light other than the excitation light which is blocked by the first cut filter and disposed adjacent to the second imaging unit on a light-incident side.

According to the second aspect of the present invention, the endoscope apparatus is capable of observing light reflected at the examination region by the second imaging unit. Therefore, the region being examined with the endoscope apparatus can be easily determined. Accordingly, the region can be easily located, and it can be easily determined whether the region is the examination region.

For example, when using a fluorescent agent that is degraded by an enzyme present in predetermined cells in the examination region and generates fluorescence of a predetermined wavelength by excitation light and another fluorescent agent that generates fluorescence of a wavelength difference from the predetermined wavelength by excitation light, the first and second imaging units can detect the area in which the fluorescent agents have spread throughout. Consequently, when fluorescence is not detected, it can be determined whether this is because the predetermined cells are not present in the examination region or because the fluorescent agent has not spread throughout examination region. As a result, an examination of the examination region can be carried out easily.

For example, when using a fluorescent agent that is degraded by an enzyme present in predetermined cells in the examination region and generates fluorescence of a predetermined wavelength by excitation light and another fluorescent agent that is degraded by an enzyme present in other cells and generates fluorescence of a predetermined wavelength by excitation light, a plurality of predetermined cells can be examined simultaneously. Since a plurality of predetermined cells can be examined by observing the examination region once, observation of the examination region can be easily carried out.

In the above-described aspects of the present invention, the illumination unit preferably includes a switching unit configured to switch between the illumination light and the excitation light (in a plurality spectral bands) so that the illumination light and the excitation light are emitted in sequence.

In this way, the beams of light reflected at the examination region and the fluorescence (in a plurality spectral bands) generated at the examination region are incident on the imaging unit or the first and second imaging units in sequence. Therefore, images of the examination region corresponding to the reflected light and the fluorescence can be obtained in sequence. As a result, image processing for the images corresponding to the reflected light and the fluorescence can be easily carried out separately. Moreover, the images of the examination region corresponding to light in a plurality of spectral bands can be captured by a single imaging unit.

In the above structure, it is preferable that the illumination unit include a visible light emission unit configured to emit illumination light and an excitation light emission unit configured to emit excitation light.

In this way, the illumination light and the excitation light are emitted from separate emission units and, thus, the emission timings of the illumination light and the excitation light can be controlled separately.

In the above-described first aspect of the present invention, it is preferable that the cut filter blocks the excitation light in the plurality of spectral bands and transmits fluorescence in at least one of the fluorescence bands. At least one of the fluorescence bands is a spectral band located between the spectral bands of the excitation light.

In this way, the cut filter blocks excitation light in the plurality of spectral bands. Moreover, at least one of the fluorescence bands of the generated fluorescence is located between the spectral bands being blocked by the filters.

Therefore, even if the wavelength of fluorescence excited by excitation light is close to the wavelength of the excitation light, the cut filter is capable of blocking the excitation light and transmitting the fluorescence.

In the above-described second aspect of the present invention, it is preferable that at least one of the first cut filter and the second cut filter block the excitation light in a plurality of spectral bands and transmit fluorescence in at least one of the fluorescence bands. At least one of the fluorescence bands is a spectral band located between the spectral bands of the excitation light.

In this way, at least one of the first cut filter and the second cut filter block excitation light in a plurality of spectral bands. Moreover, at least one of the fluorescence bands of the generated fluorescence is located between the spectral bands being blocked by the filters.

Therefore, even if the wavelength of fluorescence excited by excitation light is close to the wavelength of the excitation light, the cut filter is capable of blocking the excitation light and transmitting the fluorescence.

In the above-described first aspect of the present invention, it is preferable that the cut filter blocks light in a range of wavelengths corresponding to a single, uninterrupted spectral band.

In this way, the cut filter blocks light in a range of wavelengths that covers the spectral bands of the excitation light. Consequently, the cut filter can be produced easier than a cut filter that blocks light in a plurality of ranges of wavelengths corresponding to a plurality of spectral bands.

In the above-described second aspect of the present invention, it is preferable that at least one of the first cut filter and the second cut filter block light in a range of wavelengths corresponding to a single, uninterrupted spectral band.

In this way, at least one of the first cut filter and the second cut filter blocks light in a range of wavelengths that covers the spectral bands of the excitation light. Consequently, the cut filter can be produced easier than a cut filter that blocks light in a plurality of ranges of wavelengths corresponding to a plurality of spectral bands.

In the above aspects of the present invention, it is preferable that the endoscope apparatus further include a fluorescence determination unit for determining whether fluorescence in a predetermined fluorescence band is generated in a predetermined region of the body to be examined on the basis of a fluorescence image captured by the imaging unit.

In this way, the endoscope apparatus can determine whether predetermined fluorescence is generated in a predetermined region of the examination region by the fluorescence determination unit. Therefore, it can be determined whether the plurality of fluorescent agents has spread throughout the predetermined region.

According to a third aspect of the present invention, a method for determining whether fluorescence is generated using an endoscope apparatus includes the steps of emitting excitation light in a plurality of spectral bands from an insertion section of an endoscope apparatus into a body to be examined, blocking the excitation light reflected at the body to be examined while capturing an image corresponding to fluorescence in a plurality of spectral bands generated inside the body to be examined, and determining whether predetermined fluorescence has been generated in a predetermined region of the body to be examined on the basis of the fluorescence images captured in the blocking step.

According to the third aspect of the present invention, whether a plurality of fluorescent agents administered to the body to be examined has spread throughout a predetermined region can be determined by determining whether predetermined fluorescence is generated at the predetermined region of the body to be examined.

In the third aspect of the present invention, it is preferable that the determining step include the steps of measuring the amount of fluorescence in one of the fluorescence bands among the plurality of fluorescence bands generated at the predetermined region, determining whether fluorescent agents used to generate the fluorescence in the plurality of fluorescence bands are spread throughout the predetermined region on the basis of the amount of fluorescence, and detecting fluorescence in another fluorescence band after the fluorescent agents are determined to be spread throughout the predetermined region in the determining step.

In this way, it can be determined whether the amount of fluorescence generated at the predetermined region in the body to be examined exceeds a predetermined amount. When the amount of fluorescence does not exceed the predetermined amount, it is determined that none of the fluorescent agents are spread throughout the predetermined region in the examination region. Contrastingly, when the amount of fluorescence exceeds the predetermined amount, it is determined that the fluorescent agents are spread throughout the predetermined region and other fluorescence is detected.

When other fluorescence is not detected, it can be determined that the fluorescent agents are spread throughout the body to be examined and the amount of the fluorescent agent that exists in predetermined cells does not exceed a predetermined amount.

### BRIEF DESCRIPTION OF THE SEVERAL VIEWS OF THE DRAWINGS

FIG. 1 is a schematic view of an endoscope apparatus according to a first embodiment of the present invention.

FIG. 2 is plan view illustrating an illumination filter included in a light source unit shown in FIG. 1.

FIGS. 3A to 3C illustrate the relationship between the transmittance of each filter of the illumination filter, the wavelengths of excitation light and fluorescence generated by fluorescent agents, and the transmittance of a cut filter.

FIG. 4 illustrates an example of an image of a lesion examined with the endoscope apparatus shown in FIG. 1.

FIG. 5 illustrates another example of an image of a lesion examined with the endoscope apparatus shown in FIG. 1.

FIG. 6 is a schematic view of an endoscope apparatus according to a modification of the first embodiment.

FIG. 7 is plan view of an illumination filter provided at a light source unit shown in FIG. 6.

FIG. 8 is a schematic view of an endoscope apparatus according to a second modification of the first embodiment.

FIG. 9 is a flow chart illustrating a method for controlling the endoscope apparatus shown in FIG. 8.

FIG. 10 is a schematic view of a display system of the monitor illustrated in FIG. 8.

FIG. 11 is a schematic view of another display system of the monitor illustrated in FIG. 8.

FIG. 12 is a schematic view of an endoscope apparatus according to a second embodiment of the present invention.

FIGS. 13A to 13C illustrate the wavelengths of excitation light and fluorescence generated by fluorescent agents and the transmittance of a cut filter.

FIG. 14 is a schematic view of an endoscope apparatus according to a third embodiment of the present invention.

FIGS. 15A to 15D illustrate the wavelengths of excitation light and fluorescence generated by fluorescent agents and the light transmittance of a cut filter.

### DETAILED DESCRIPTION OF THE INVENTION

### First Embodiment

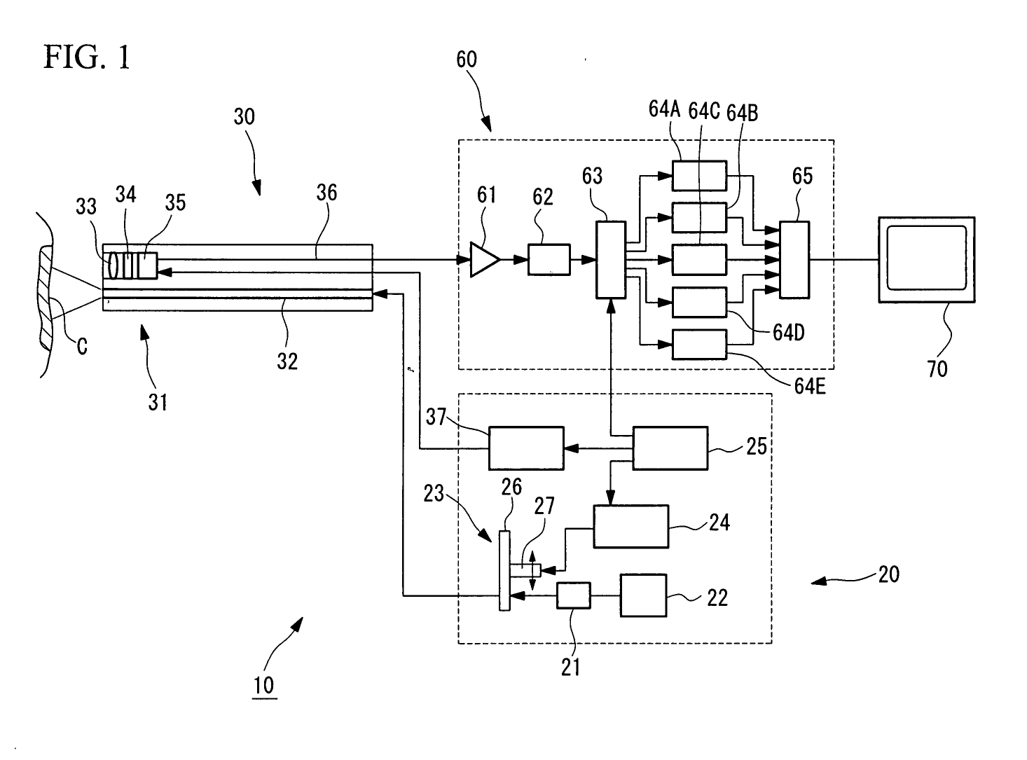

An endoscope apparatus according to a first embodiment of the present invention will be described below with reference to FIGS. 1 to 5.

An endoscope apparatus 10, illustrated in FIG. 1, is an endoscope apparatus capable of examining a lesion by using fluorescence.

The endoscope apparatus 10, as illustrated in FIG. 1, includes a light source unit (illumination unit) 20 that emits beams of visible light and excitation light in sequence, an endoscope (insertion section) 30 that is to be inserted in a body cavity for capturing an image of a lesion (examination region) C, a signal processing unit 60 for processing image signals of the lesion C (examination region, predetermined range) captured by the endoscope 30, and a monitor 70 for displaying an output from the signal processing unit 60.

The light source unit 20 includes a light source 21, such as a xenon lamp, that emits light including light in spectral bands corresponding to visible light and excitation light, a power supply 22 that supplies electrical power to the light source 21, an illumination filter 23 that converts the light emitted from the light source 21 into visible light and excitation light, a motor driver (switching unit) 24 that rotationally drives the illumination filter 23, and a timing generator (switching unit) 25 that controls the rotational speed and phase of the illumination filter 23.

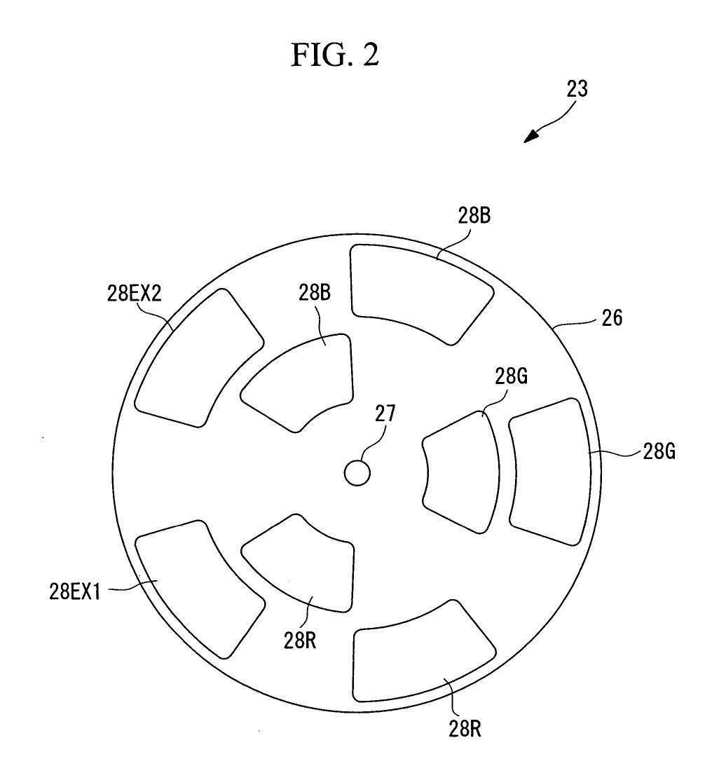

FIG. 2 is plan view illustrating the structure of the illumination filter 23 included in the light source unit 20.

The illumination filter 23, as illustrated in FIG. 2, includes a disk-shaped planar member 26 and a rotary shaft 27 provided at the center of the planar member 26.

The rotary shaft 27 is connected to a motor (not shown in the drawing) controlled by the motor driver 24.

Groups of filters are provided around the inner and outer circumferences of the planar member 26. Around the outer circumference, a blue light filter (visible light illumination unit) 28B that transmits blue light (illumination light) B, a green light filter (visible light illumination unit) 28G that transmits green light (illumination light) G, a red light filter (visible light illumination unit) 28R that transmits red light (illumination light) R, a first excitation filter (excitation light illumination unit) 28EX1 that transmits first excitation light (excitation light) EX1 in an infrared band, and a second excitation filter (excitation light illumination unit) 28EX2 that transmits second excitation light (excitation light) EX2 having a wavelength longer than the first excitation light are provided. The filters 28B, 28G, 28R, 28EX1, and 28EX2 are disposed equal distances apart from each other.

Around the inner circumference, a blue filter 28B, a green filter 28G, and a red light filter 28R are disposed. The filters 28B, 28G, and 28R are disposed equal distances apart from each other.

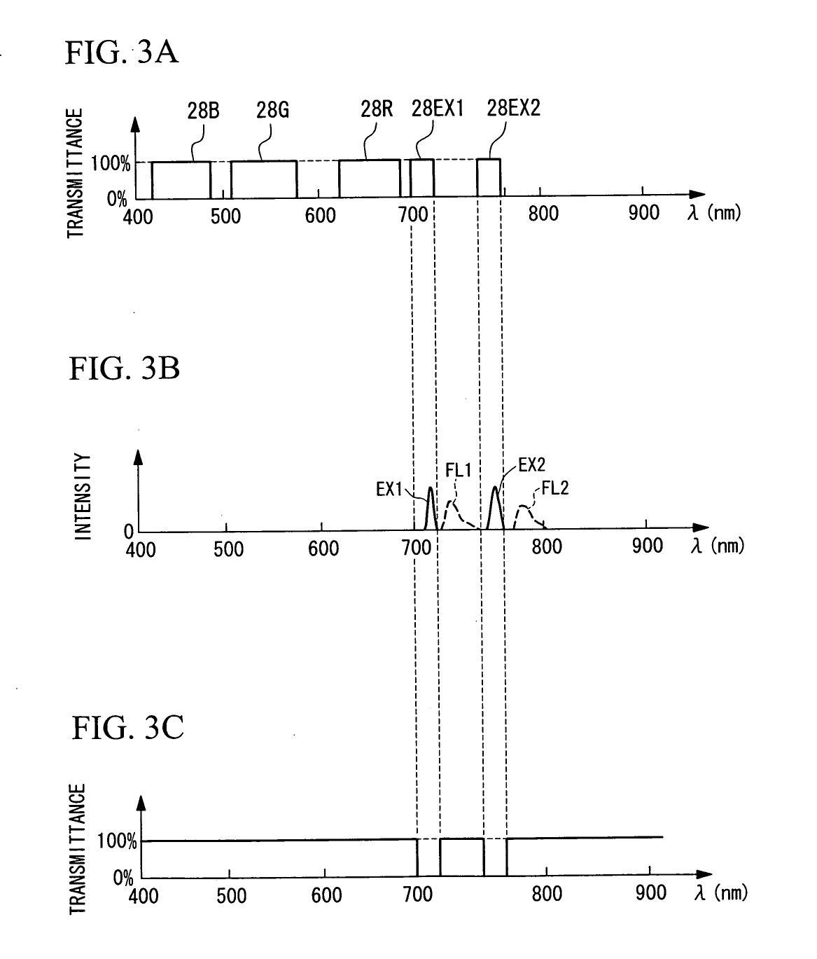

FIG. 3A shows the transmittance of each filter included in the illumination filter 23. As shown in Graph i, the wavelengths of the light transmitted through the blue filters 28B, the green filters 28G, the red filters 28R, the first excitation filter 28EX1, and the second excitation filter 28EX2 increase in this order.

The light transmitted through the blue light filters 28B, the green light filters 28G, and the red light filters 28R is in a spectral band corresponding to visible light, and the light transmitted through the first excitation filter 28EX1 and the second excitation filter 28EX2 is in the infrared band.

The transmittances of the blue light filters 28B, the green light filters 28G, and the red light filters 28R disposed around the inner and outer circumferences of the illumination filter 23 may be the same or different.

For example, the overall balance of the transmittances of the blue light filters 28B, the green light filters 28G, and the red light filters 28R disposed around the inner circumference may be shifted toward longer wavelengths. Since the group of filters disposed around the inner circumference does not include filters that transmit excitation light in the infrared band, the overall balance of the illumination light in the visible spectral band can be shifted, as mentioned above, toward longer wavelengths.

The illumination filter 23 is disposed on a movable member (not shown) so that the illumination filter 23 moves parallel to the plane of the planar member 26 (for example, in the vertical direction in FIG. 2). In this way, the area where light from the light source 21 is incident can be switched between an area at the inner circumference of the planar member 26 and an area at the outer circumference of the planar member 26. For example, by radiating light from the light source 21 on an area at the inner circumference of the planar member 26, R spectral-band light, G spectral-band light, and B spectral-band light are emitted in sequence from the light source unit 20. Accordingly, the lesion C can be examined by being illuminated only with visible light. By radiating light from the light source 21 on an area at the outer circumference of the planar member 26, the R, G, and B spectral-band light, first excitation light EX1, and second excitation light EX2 can be emitted in sequence from the light source unit 20.

The timing generator 25 generates timing signals that control the B, G, R, EX1, and EX2 spectral-band light so that each type of light is emitted in sequence. The timing generator 25 is connected to the motor driver 24 SO that timing signals can be sent to the motor driver 24. The timing generator 25 is also connected to a CCD driver 37 that controls a CCD 35, described below, so that the generated timing signals are sent to the CCD driver 37. Moreover, the timing generator 25 is connected to a switching unit 63 of the signal processing unit 60, described below, so that the timing signals can be output to the switching unit 63.

The endoscope 30 has a light guide 32 for guiding each type of spectral-band light emitted from the light source unit 20 to a distal end 31 of the endoscope 30. The distal end 31 includes a lens system 33 that receives reflected light and fluorescence from the lesion C, a cut filter 34 that blocks the first excitation light EX1 and the second excitation light EX2 included in the light transmitted through the lens system 33, and the CCD (imaging unit) 35 where an image of the lesion C is formed by the light transmitted through the cut filter 34.

The image of the lesion C captured by the CCD 35 is sent to the signal processing unit 60 as image signals corresponding to the R, G, B, EX1, and EX2 spectral-band light via a line 36 connected to the endoscope 30.

The signal processing unit 60 includes an amplifier 61 for amplifying an image signal, an analog/digital (A/D) converter 62 for converting image signals into digital signals, the switching unit 63 for directing digital signals to the subsequent memories, a first memory 64A, a second memory 64B, a third memory 64C, a fourth memory 64D, and a fifth memory 64E for temporarily storing digital signals, and a digital/analog (D/A) converter 65 for converting digital signals from the memories into analog image signals and for sending the converted analog image signals to the monitor 70.

The switching unit 63 directs the digital image signals of the lesion C corresponding to the R, G, B, EX1, and EX2 spectral-band light on the basis of timing signals sent from the timing generator 25.

Next the operation of the endoscope apparatus 10 having the above-described structure will be described.

When electrical power is supplied from the power supply 22 to the light source 21, light including the R, G, B, EX1, and EX2 spectral-band light is emitted to the illumination filter 23, as illustrated in FIGS. 1 and 2. The illumination filter 23 rotates around the rotary shaft 27, and, for example, when light from the light source 21 is radiated on an area at the outer circumference of the illumination filter 23, light from the light source 21 is incident on the filters 28B, 28G, 28R, 28EX1, and 28EX2 in sequence.

The B, G, R, EX1, and EX2 spectral-band light emitted from the filters 28B, 28G, 28R, 28EX1, and 28EX2, respectively, is emitted in sequence in accordance with the rotational cycle of the illumination filter 23. The B, G, R, EX1, and EX2 spectral-band light that is transmitted through the illumination filter 23 is incident on the light guide 32 of the endoscope 30. The B, G, R, EX1, and EX2 spectral-band light is then guided through the light guide 32 and is introduced into the body cavity. In this way, the lesion C is illuminated.

FIG. 3B shows the wavelengths of the excitation light used to excite the fluorescent agents administered to the lesion C and the fluorescence generated by the fluorescent agents. A first fluorescent agent that generates first fluorescence FL1 when irradiated with the first excitation light EX1 and a second fluorescent agent that is degraded by an enzyme present in the cells of the lesion C (for example, cancer cells) and that generates second fluorescence FL2 when irradiated with the second excitation light EX2 are injected in the lesion C.

In this way, the first fluorescence FL1 is generated by the first fluorescent agent spread throughout a region in the lesion C, and the second fluorescence FL2 is generated by the second fluorescent agent spread throughout the cancer cells.

The fluorescent agents injected into the lesion C, as described above, may be two different fluorescent agents: a fluorescent agent that generates fluorescence when irradiated with excitation light; and a fluorescent agent that is degraded by a predetermined enzyme and generates fluorescence when irradiated with excitation light. Instead, the same fluorescent agents that generate fluorescence only when irradiated with excitation light may be injected into the lesion C. The types and combination of the fluorescent agents are not limited.

Fluorescent agent that generates fluorescence in the infrared band, as described above, are Cy5, Cy7, IRD 700, and Lafolla Blue, for example.

The B, G, R, EX1, and EX2 spectral-band light reflected from the lesion C and the fluorescence FL1 and FL2 generated by the fluorescent agent are transmitted through the lens system 33 and are incident on the cut filter 34, as illustrated in FIG. 1.

The cut filter 34, as shown in FIG. 3C, blocks light in the same spectral bands as the excitation light EX1 and EX2 and transmits light in the other spectral bands. Thus, the B, G, and R spectral-band light reflected from the lesion C and the fluorescence FL1 and FL2 generated at the lesion C are transmitted through the cut filter 34 and are incident on the CCD 35.

Under the control of the CCD driver 37, the CCD 35 generates image signals (analog signals) corresponding to the images formed by the B, G, and R spectral-band light, the first fluorescence FL1, and the second fluorescence FL2, which enter the CCD 35 in sequence. The generated image signals are sent to the signal processing unit 60 in the same order in which they are generated.

First, the image signals input to the signal processing unit 60 are amplified by the amplifier 61. Then, the image signals are converted into digital signals by the A/D converter 62 and are sent to the switching unit 63.

The switching unit 63 directs the digital signals, which are input in sequence, to the memories 64A, 64B, 64C, 64D, and 64E on the basis of the timing signal from the timing generator 25. More specifically, a digital image signal of the lesion C corresponding to the blue light B is directed to the first memory 64A by the switching unit 63 and is temporarily stored in the first memory 64A. Similarly, a digital image signal corresponding to the green light G is directed to the second memory 64B, and a digital image signal corresponding to the red light R is directed to the third memory 64C. The digital signal of the fluorescence image corresponding to the first fluorescence FL1 is directed to the fourth memory 64D by the switching unit 63, and the digital signal of the fluorescence image corresponding to the second fluorescence FL2 is directed to the fifth memory 64E.

The digital image signals stored in the memories 64A, 64B, 64C, 64D, and 64E are output to the D/A converter 65. The digital image signals input to the D/A converter 65 are converted into analog image signals, and the analog image signals are output to the monitor 70.

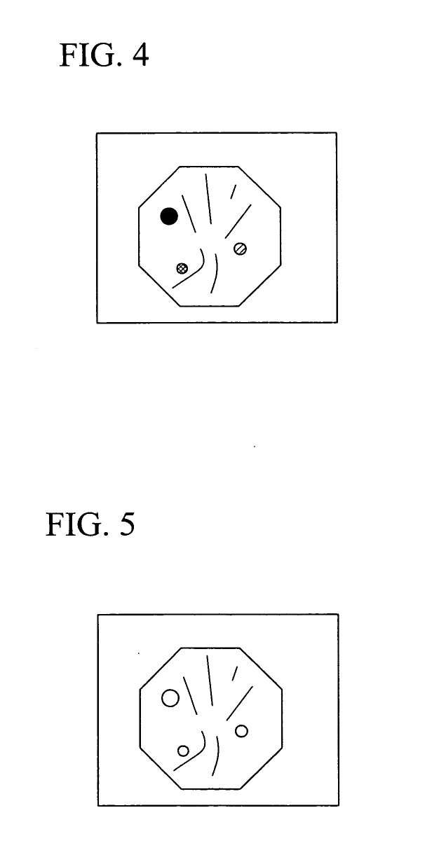

The images of the lesion C corresponding to the B, G, and R spectral-band light are recombined and are displayed on the monitor 70 as a color image of the lesion C. The images of the first fluorescence FL1 and the second fluorescence FL2 are superimposed on the color image of the lesion C, as illustrated in FIG. 4. The intensity of the fluorescence may be represented by the density of the color used to display the fluorescence image or may be represented by the hue of the color used to display the fluorescence image (for example, as the intensity of the fluorescence becomes greater, the color used to display the fluorescence image may gradually change from blue to red). As illustrated in FIG. 5, only the outlines of the fluorescence images may be displayed.

The observer may switch the method of displaying the image of the lesion C by providing an input. The method may be switched between one in which the fluorescence image is superimposed on the color image and one in which the color image and the fluorescence image are displayed side by side. The observer may select one of the images of the first fluorescence FL1 and FL2 to be displayed, and, moreover, the observer may switch the image to be displayed by providing an input.

According to the above-described structure, the fluorescence FL1 and FL2 in two difference spectral bands can be observed simultaneously. By observing the first fluorescence FL1, when the second fluorescence FL2 is not detected, it can be determined whether this is because cancer cells are not present in the lesion C or because the second fluorescent agent has not spread throughout the lesion C. As a result, examination of the lesion C can be carried out easily.

Since beams of B, G, R, EX1, and EX2 spectral-band light are emitted in sequence from the light source unit 20, the beams of B, G, R, EX1, and EX2 spectral-band light reflected from the lesion C enter the CCD 35 in sequence. Therefore, the beams of spectral-band light making up the images can be converted into separate image signals by using a single CCD 35. Consequently, the size of the distal end 31 of the endoscope 30 can be easily reduced.

Since B, G, and R light in the visible spectral band can be observed using the endoscope apparatus 10, the image observed using the endoscope apparatus 10 can be displayed as a color image. Therefore, the site being examined with the endoscope apparatus 10 can be easily determined. Accordingly, the lesion C can be easily located, and it can be easily determined whether the site being examined is the lesion C.

### First Modification of First Embodiment

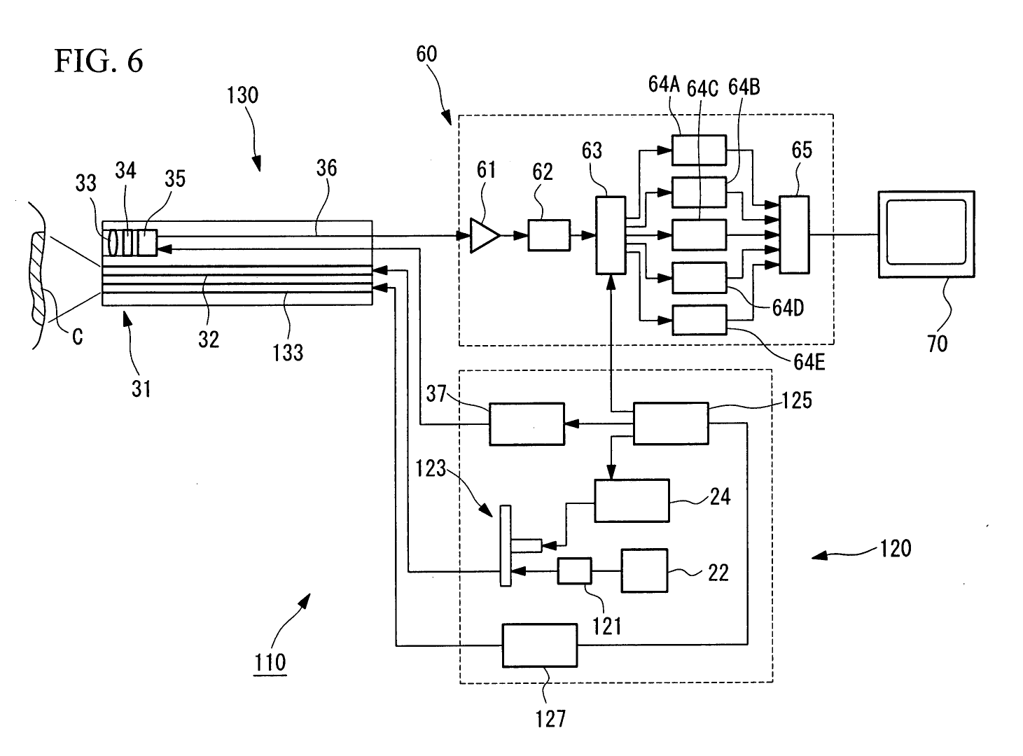

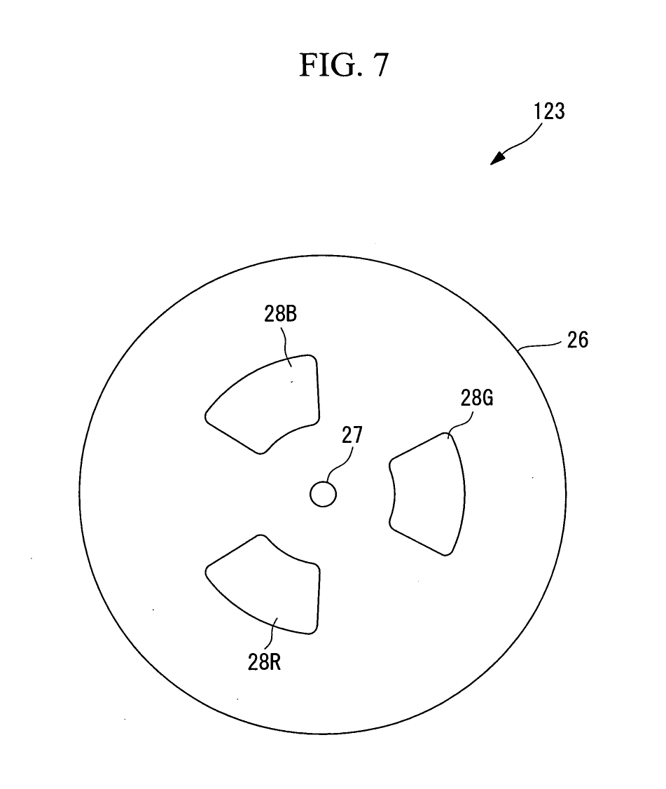

Next a first modification of the first embodiment will be described with reference to FIGS. 6 and 7.

The basic structure of the endoscope apparatus according to this modification is the same as that of the endoscope apparatus 10 according to the first embodiment except that the structures of the light source unit and the endoscope differ. Only the components related to the light source unit and the endoscope of the endoscope apparatus according to this modification will be described with reference to FIGS. 6 and 7, and descriptions of other components, such as the signal processing unit, will be omitted.

Components that are the same as those included in the endoscope apparatus according to the first embodiment are represented by the same reference numerals.

FIG. 6 illustrates an endoscope apparatus 110 according to the first modification of the first embodiment of the present invention. The endoscope apparatus 110 is capable of examining a lesion by detecting fluorescence.

The endoscope apparatus 110, as illustrated in FIG. 6, includes a light source unit (illumination unit) 120 that emits beams of visible light and excitation light in sequence, an endoscope (insertion section) 130 that is to be inserted in a body cavity for capturing an image of a lesion C, a signal processing unit 60 for processing image signals of the lesion C captured by the endoscope 130, and a monitor 70 for displaying an output from the signal processing unit 60.

The light source unit 120 includes a light source 121, such as a xenon lamp, that emits light including light in spectral bands corresponding to visible light, a power supply 22 that supplies electrical power to the light source 121, an illumination filter (visible light illumination unit) 123 that converts the light emitted from the light source 121 into B, G, and R spectral-band light, a motor driver 24 that rotationally drives the illumination filter 123, a laser source (excitation light illumination unit) 127 that emits first excitation light EX1 and second excitation light EX2, and a timing generator (switching unit) 125 that controls the rotational speed and phase of the illumination filter 123.

FIG. 7 is a plan view illustrating the structure of the illumination filter 123 included in the light source unit 120.

The illumination filter 123, as illustrated in FIG. 7, includes a disk-shaped planar member 26 and a rotary shaft 27 provided at the center of the planar member 26.

A group of filters is provided the circumference of the planar member 26. Around the circumference, a blue light filter 28B, a green light filter 28G, and a red light filter 28R are disposed. By employing such a structure, first excitation light EX1 and second excitation light EX2 can be emitted during the period between emission of the red light R and emission of the blue light B.

The laser source 127 emits first excitation light EX1 and second excitation light EX2 during the period between the emission of the red light R and the emission of the blue light B on the basis of timing signals input from the timing generator 25.

The first excitation light EX1 and EX2 may be emitted in an order from the excitation light having a longer wavelength to the excitation light having a shorter wavelength, or in the opposite order, that is, from the excitation light having a shorter wavelength to the excitation light having a longer wavelength. The emission sequence is not limited, however.

The endoscope 130 has a light guide 32 for guiding B, G, and R spectral-band light emitted from the illumination filter 123 to a distal end 31 of the endoscope 130 and an excitation light guide 133 for guiding the excitation light EX1 and EX2 emitted from the laser source 127 to the distal end 31.

Next, the operation of the endoscope apparatus 110 having the above-described structure will be described.

When electrical power is supplied from the power supply 22 to the light source 121, light including the R, G, and B, spectral-band light is emitted to the illumination filter 123, as illustrated in FIG. 6. The illumination filter 123 rotates around the rotary shaft 27 So that light from the light source 21 is incident on the filters 28B, 28G, and 28R in sequence.

The B, G, and R spectral-band light emitted from the filters 28B, 28G, and 28R, respectively, is emitted in sequence in accordance with the rotational cycle of the illumination filter 123. The first excitation light EX1 and the second excitation light EX2 are emitted in sequence from the laser source 127. The illumination filter 123 and the laser source 127 are controlled on the basis of timing signals from the timing generator 25. Thus, the beams of B, G, R, EX1, and EX2 spectral-band light can be emitted in sequence from the light source unit 120.

The B, G, and R spectral-band light transmitted through the illumination filter 123 enters the light guide 32 of the endoscope 30. The B, G, and R spectral-band light is guided and emitted to a body cavity through the light guide 32. In this way, the lesion C is illuminated. The first excitation light EX1 and the second excitation light EX2 emitted from the laser source 127 enters the excitation light guide 133. The excitation light EX1 and EX2 is guided and emitted to the body cavity through the excitation light guide 133. In this way, the lesion C is illuminated.

Since the steps of the operation after this are the same as those according to the first embodiment, descriptions thereof are omitted.

According to the above-described structure, the illumination filter 123 that transmits the illumination light B, G, and R in the visible spectral and the laser source 127 that generates the excitation light EX1 and EX2 in the infrared band are provided separately. In this way, the emission timing of the beams of B, G, and R light and the emission timing of the beams of excitation light EX1 and EX2 can be controlled independently.

### Second Modification of First Embodiment

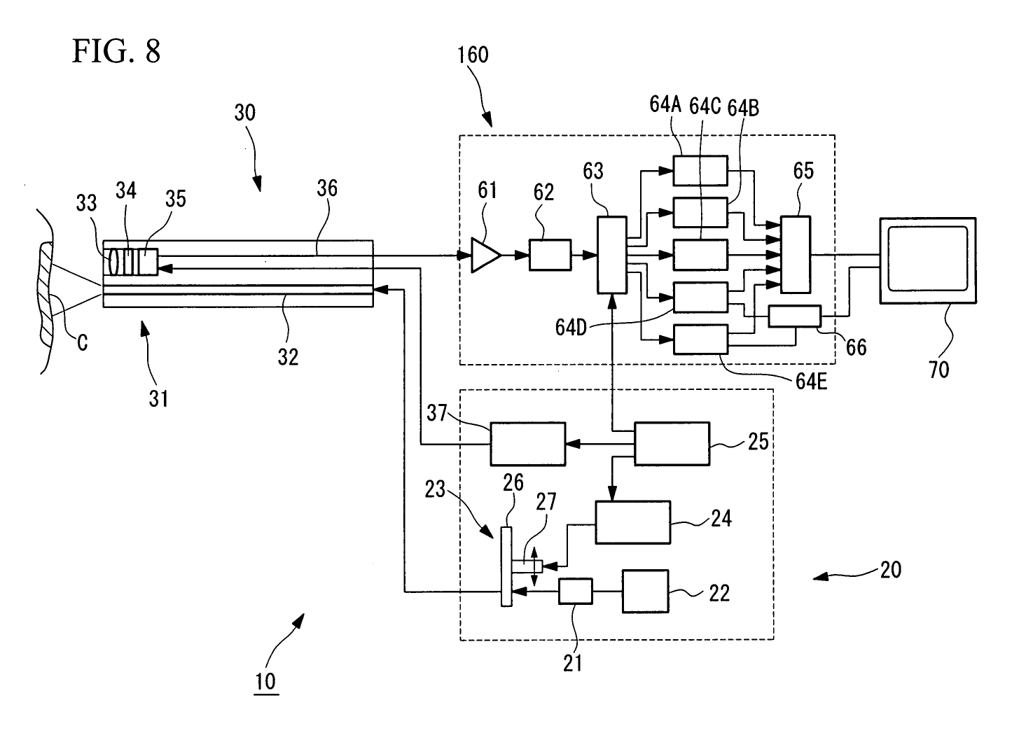

Next, a second modification of the first embodiment will be described with reference to FIGS. 8 to 11.

The basic structure of the endoscope apparatus according to this modification is the same as that of the endoscope apparatus 10 according to the first embodiment except that the inner structure of the signal processing unit 60 differs. Only the components related to the signal processing unit 60 according to this modification will be described with reference to FIGS. 8 to 11, and descriptions of other components, such as the endoscope, will be omitted.

Components that are the same as those included in the endoscope apparatus according to the first embodiment are represent by the same reference numerals.

As illustrated in FIG. 8, a signal processing unit 160 includes an amplifier 61 that amplifies image signals, an analog/digital (A/D) converter 62 that converts image signals into digital signals, subsequent memories, a first memory 64A, a second memory 64B, a third memory 64C, a fourth memory 64D, and a fifth memory 64E for temporarily storing digital signals, a digital/analog (D/A) converter 65 that converts digital signals output from the memories into analog image signals and sends the converted analog image signals to a monitor 70, and a fluorescence signal measurement unit (fluorescence detection unit) 66.

The switching unit 63 directs the digital image signals of the lesion C corresponding to R, G, B, EX1, and EX2 spectral-band light on the basis of timing signals sent from the timing generator 25.

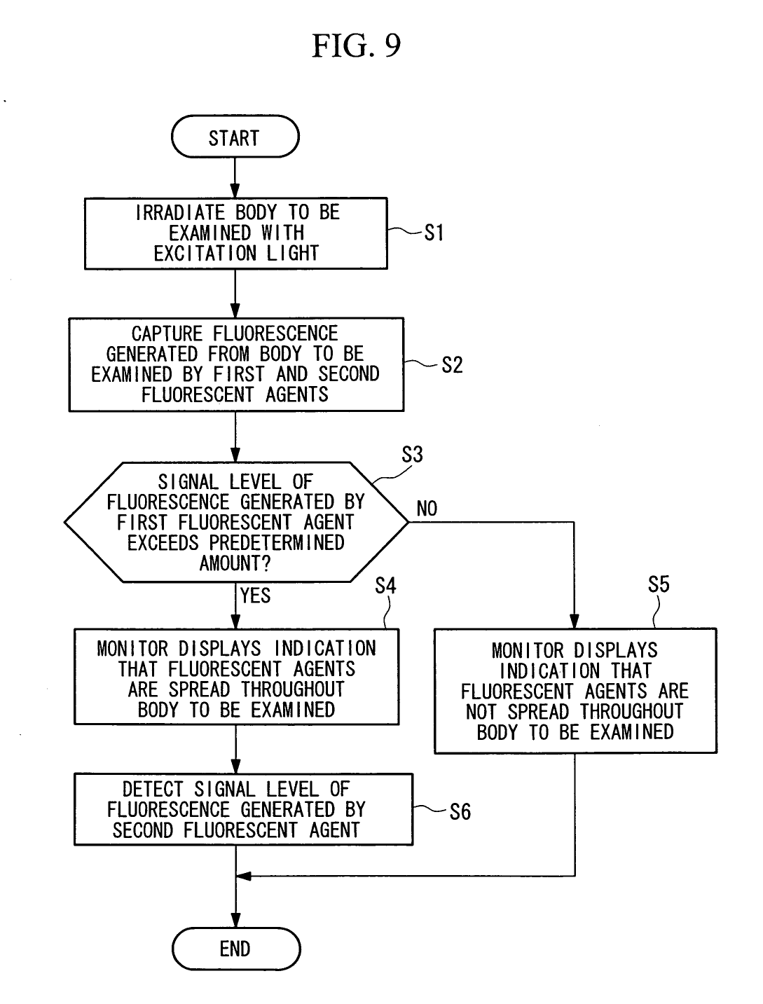

Next, a method for determining whether fluorescent agents have spread throughout the lesion C will be described with reference to the flow chart in FIG. 9.

The excitation light EX1 and EX2 guided into the body cavity through a light guide 32 are radiated on the lesion C (illuminating step, Step S1). First fluorescence FL1 and second fluorescence FL2 generated at the lesion (body to be examined) C are transmitted through a cut filter 34 and are incident on a CCD 35. The CCD 35 captures the fluorescence FL1 and FL2 (capturing step, Step S2).

The analog signals corresponding to the fluorescence FL1 and FL2 generated at the CCD 35 are converted into digital signals and are sent to the signal processing unit 60. Then, the digital signals are directed to the memories 64D and 64E by the switching unit 63.

The fluorescence signal measurement unit 66 reads out digital image signals corresponding to the first fluorescence FL1 stored in the fourth memory 64D and calculates the total fluorescence signal level of the first fluorescence FL1 corresponding to all pixels (measuring step). Then, the fluorescence level is higher or lower than a predetermined value (determining step, Step S3).

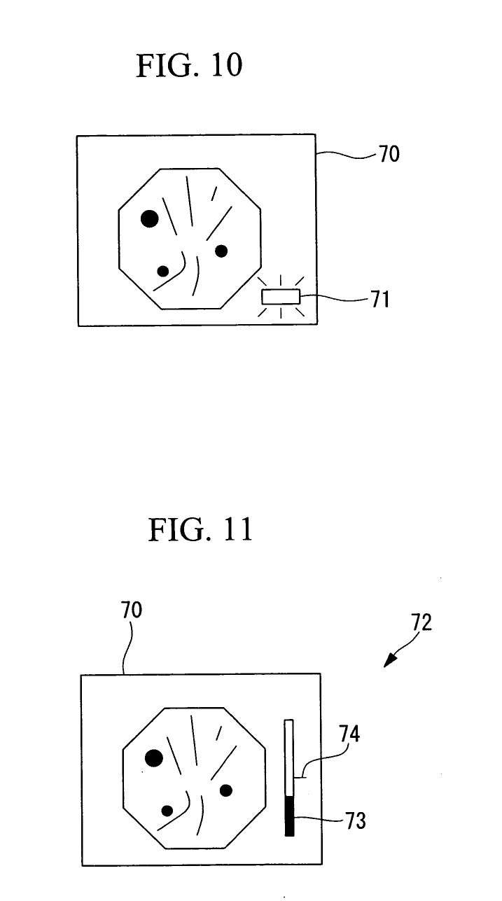

If the total signal level is higher than a predetermined value, a blinking marker (fluorescence determination unit) 71 is displayed on the monitor 70 (refer to FIG. 10) to indicate that the fluorescent agents have spread throughout the lesion C (Step S4).

If the total signal level is lower than the predetermined value, the indicator 71 on the monitor 70 is turned off to indicate that the fluorescent agents have not spread throughout the lesion C (Step S5).

If the fluorescent agents have spread throughout the lesion C, digital image signals corresponding to the second fluorescence FL2 stored in the fifth memory 64E are read out and the fluorescence signal level of the second fluorescence FL2 is detected (detecting step, Step S6). If the second fluorescence FL2 is detected, the regions generating the second fluorescence FL2 are displayed on the monitor 70 with a color different from that used for normal tissue.

As described above, if the total signal level is higher than a predetermined value, the indicator 71 on the monitor 70 may be displayed, and if the total signal level is lower than a predetermined value, the indicator 71 on the monitor 70 may be displayed.

Instead of illuminating an indicator on the monitor 70, an alarm may be sounded to notify the observer that the total signal level is higher than a predetermined value.

As described above, the amount of fluorescence FL1 may be indicated by illuminating a indicator on the monitor 70 or, as illustrated in FIG. 11, it may be indicated using a fluorescence display bar (fluorescence determination unit) 72.

The fluorescence display bar 72 includes a display bar 73 that moves vertically in proportion with the amount of fluorescence FL1 and a reference line 74 indicating a predetermined value. When the display bar 73 is above the reference line 74, this indicates that fluorescent agents have sufficiently spread throughout the lesion C. In contrast, when the display bar 73 is below the reference line 74, this indicates that the fluorescent agents have not sufficiently spread throughout the lesion C or the detected amount of fluorescence FL1 is small because the distance from the body to be examined to the endoscope apparatus 10 is too far. If the display bar 73 is zero, the fluorescent agents have not spread throughout the lesion C at all.

The observer may select the size of the area to be determined whether or not the first fluorescent agent has spread so that the area is a limited area in the entire region captured in the image.

According to the above-described structure, the observer can check the spreading of the first fluorescent agent throughout the lesion C by an indicator displayed on the monitor 70 while observing the second fluorescent agent.

### Second Embodiment

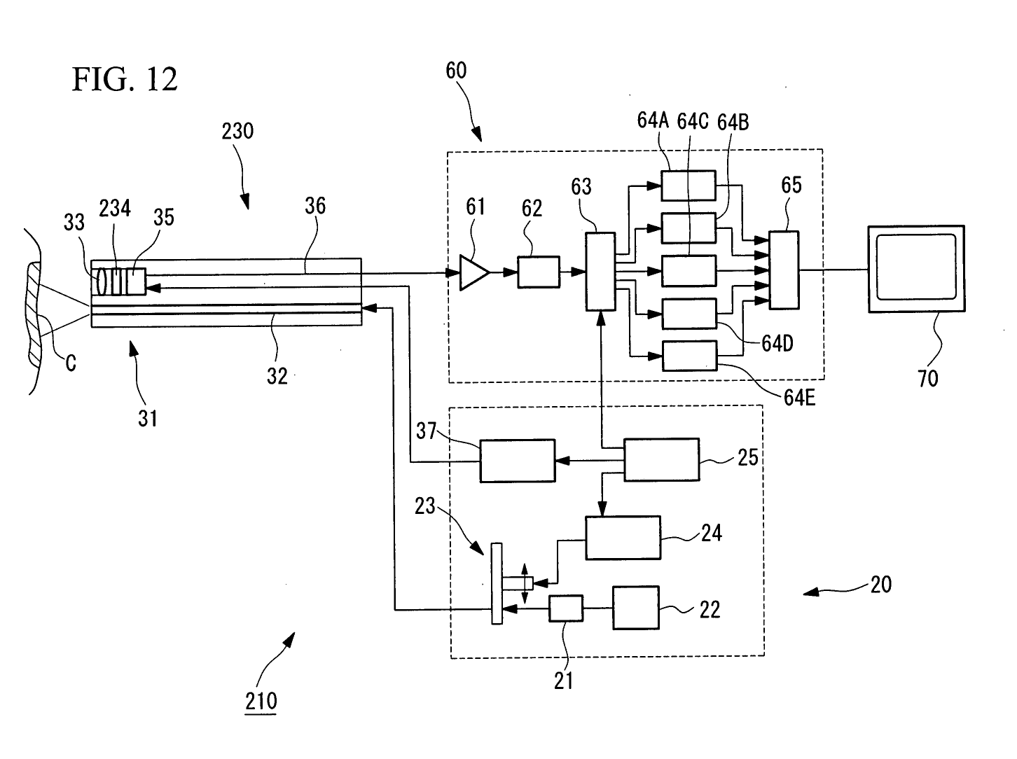

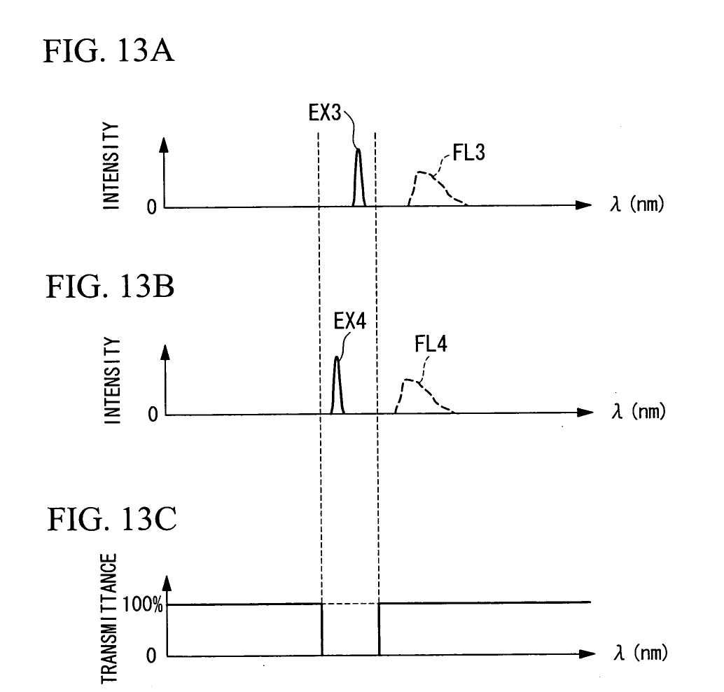

Next, a second embodiment of the present invention will be described with reference to FIGS. 12 and 13.

The basic structure of the endoscope apparatus according to this embodiment is the same as that of the endoscope apparatus 10 according to the first embodiment except that the structure of the cut filter differs. Only the components related to the cut filter of the endoscope apparatus according to this embodiment will be described with reference to FIGS. 12 and 13, and descriptions of other components, such as the signal processing unit, will be omitted.

Components that are the same as those included in the endoscope apparatus according to the first embodiment are represent by the same reference numerals.

FIG. 12 is a cross-sectional view of an endoscope apparatus 210 including an endoscope 230 according to this embodiment.

A distal end 31 of the endoscope 230 includes a lens system 33 that receives light reflected from a lesion C and fluorescence, a cut filter 234 that blocks third excitation light EX3 and fourth excitation light EX4 included in the light transmitted through the lens system 33, and a CCD 35 where an image of the lesion C is formed by the light transmitted through the cut filter 234.

FIG. 13A and FIG. 13B show the wavelengths of the excitation light EX3 and EX4 used to excite fluorescent agents introduced to the lesion C and the fluorescence generated by the fluorescent agents. A third fluorescent agent that generates third fluorescence FL3 when irradiated with the third excitation light EX3, as shown in FIG. 13A, and a fourth fluorescent agent that is degraded by an enzyme present in the cells of the lesion C (for example, cancer cells) and generates fourth fluorescence FL4 when irradiated with the fourth excitation light EX4, as shown in FIG. 13B, are injected into the lesion C.

In this way, the third fluorescence FL3 is generated by the third fluorescent agent spread throughout a region in the lesion C and the fourth fluorescence FL4 is generated by the fourth fluorescent agent spread throughout the cancer cells.

The B, G, R, EX3, and EX4 spectral-band light reflected at the lesion C and the fluorescence FL3 and FL4 generated by the fluorescent agents are transmitted through the lens system 33 and are incident on the cut filter 234, as illustrated in FIG. 12.

The cut filter 234, as shown in FIG. 13C, blocks light in the same spectral bands as the excitation light EX3 and EX4 and transmits light in the other spectral bands. Thus, the B, G, and R spectral-band light reflected at the lesion C and the fluorescence FL1 and FL2 generated at the lesion C are transmitted through the cut filter 234 and are incident on the CCD 35.

According to the above-described structure, fluorescent agents that generate fluorescence in spectral bands close to each other, such as the excitation light EX3 and EX4, can be used. Thus, the number of combinations of fluorescent agents that can be used is increased, and observation of the lesion C can be easily carried out.

### Third Embodiment

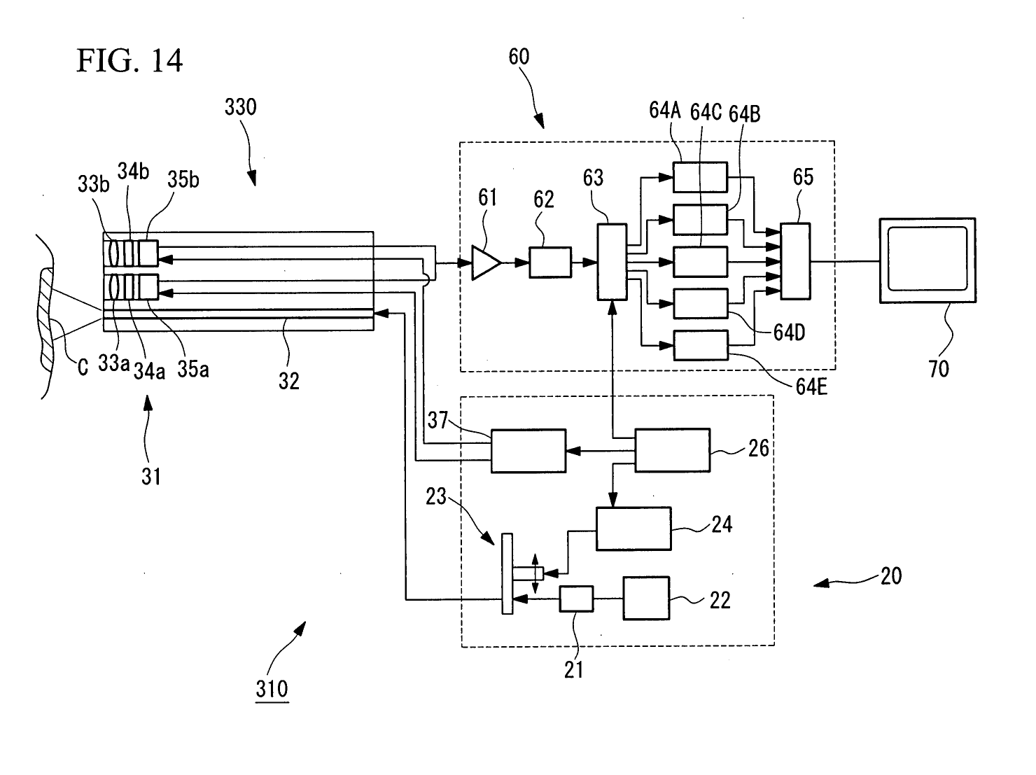

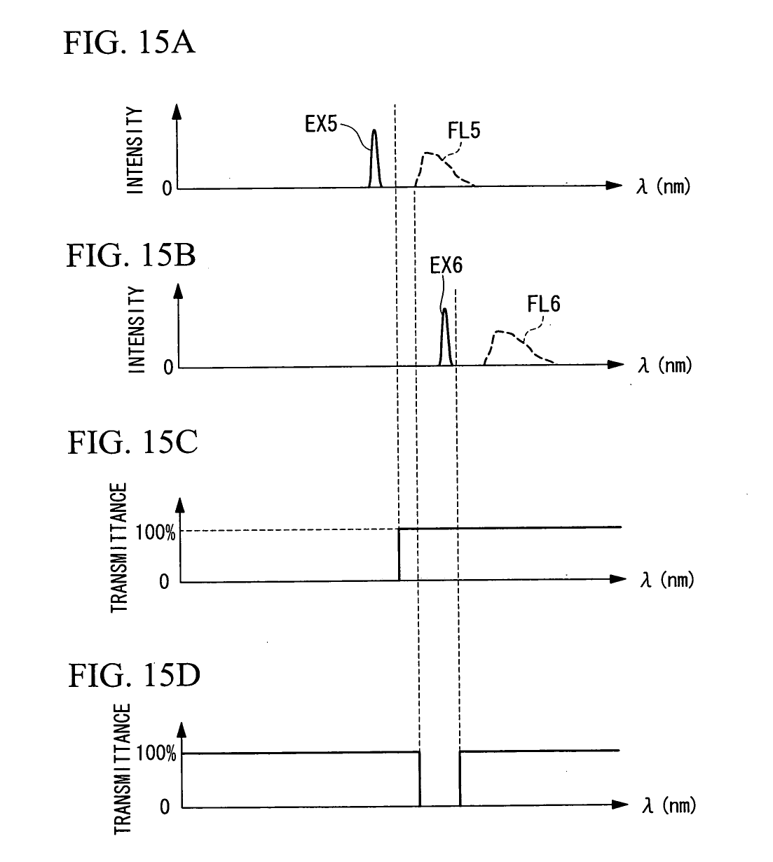

Next, a third embodiment of the present invention will be described with reference to FIGS. 14 and 15.

The basic structure of the endoscope apparatus according to this embodiment is the same as that of the endoscope apparatus 10 according to the first embodiment except that the structure of the endoscope differs. Only the components related to the endoscope of the endoscope apparatus according to this embodiment will be described with reference to FIGS. 14 and 15, and descriptions of other components, such as the signal processing unit, will be omitted.

Components that are the same as those included in the endoscope apparatus according to the first embodiment are represent by the same reference numerals.

The endoscope apparatus 310 illustrated in FIG. 14 is an endoscope apparatus configured to examine a lesion by using fluorescence.

The endoscope apparatus 310, as illustrated in FIG. 14, includes a light source unit 20 that emits beams of visible light and excitation light in sequence, an endoscope (insertion section) 330 that is to be inserted in a body cavity for capturing an image of a lesion C, a signal processing unit 60 configured to process image signals of the lesion C captured by the endoscope 330, and a monitor 70 that displays an output from the signal processing unit 60.

A distal end 31 of the endoscope 330 includes a first lens system 33a and a second lens system 33b that receive light and fluorescence from the lesion C, first and second cut filters 34a and 34b that block light having predetermined wavelengths included in the light transmitted through the lens systems 33a and 33b, respectively, and first and second CCDs (first imaging units) 35a and 35b where images of the lesion C are formed by the light transmitted through the cut filters 34a and 33b, respectively.

FIG. 15A and FIG. 15B show the wavelengths of the excitation light used to excite fluorescent agents introduced into the lesion C and the fluorescence generated by the fluorescent agents.

A fifth fluorescent agent that generates fifth fluorescence FL5 when irradiated with fifth excitation light EX5, as shown in FIG. 15A, and a sixth fluorescent agent that is degraded by an enzyme present in the cells of the lesion C (for example, cancer cells) and generates sixth fluorescence FL6 when irradiated with sixth excitation light EX6, as shown in FIG. 15B, are injected into the lesion C.

In this way, the fifth fluorescence FL5 is generated by the fifth fluorescent agent spread throughout a region in the lesion C, and the sixth fluorescence FL6 is generated by the sixth fluorescent agent spread throughout the cancer cells.

The B, G, R, EX5, and EX6 spectral-band light reflected at the lesion C and the fluorescence FL5 and FL6 generated by the fluorescent agents are transmitted through the lens systems 33a and 33b and are incident on the cut filters 34a and 34b, as illustrated in FIG. 14.

The first cut filter 34a, as shown in FIG. 15C, blocks light that has a wavelength shorter than the fifth fluorescence FL5 and transmits light that has a wavelength longer than the fifth fluorescence FL5. Consequently, the fifth fluorescence FL5, the sixth excitation light EX6, and the sixth fluorescence FL6 are incident on the first CCD 35a.

The second cut filter 34b, as shown in FIG. 15D, blocks a predetermined spectral-band light including the sixth excitation light EX6 and transmits light in the other spectral bands. Thus, the blue light B, the green light G, the red light R, the fifth excitation light EX5, and the sixth fluorescence FL6 are incident on the second CCD 35b.

As the first cut filter 34a, a filter that blocks all spectral-band light having a wavelength shorter than the fifth fluorescence FL5 may be used, as described above, or a filter similar to the second cut filter 34b that only blocks a predetermined spectral-band light including the fifth excitation light EX5 may be used.

The cut filters 34a and 34b, as described above, may be filters that transmit light in the entire visible spectral band or may be filters that only transmit light in spectral bands corresponding to blue light B, green light G, and red light R.

Next, the operation of the endoscope apparatus 310 having the above-described structure will be described.

Since the process up to the step in which the B, G, R, EX5, and EX6 spectral-band light reflected at the lesion C and the fluorescence FL5 and FL6 are incident on the cut filters 34a and 34b is the same as that according to the first embodiment, a description thereof is omitted.

The beams of the fifth fluorescence FL5, the sixth excitation light EX6, and the sixth fluorescence FL6 that have been transmitted through the first cut filter 34a are incident on the first CCD 35a in sequence. The first CCD 35a is controlled by a CCD driver 37 operating on the basis of timing signals so that only the fifth fluorescence FL5 is captured.

The beams of the blue light B, the green light G, the red light R, the fifth excitation light EX5, and the sixth fluorescence FL6 that have been transmitted through the second cut filter 34b are incident on the second CCD 35b in sequence. The second CCD 35b is controlled by the CCD driver 37 operating on the basis of timing signals so that only the blue light B, the green light G, the red light R, and the sixth fluorescence FL6 are captured.

The image of the fifth fluorescence captured by the first CCD 35a is converted into an image signal and is sent to an amplifier 61.

The images of the lesion C by the blue light B, the green light G, and the red light R and the image of the sixth fluorescence FL6 captured by the second CCD 35b are converted into image signals and are sent to the amplifier 61.

Since the subsequent process is the same as that of the first embodiment, a description thereof is omitted.

According to the above-described structure, the fifth fluorescence FL5 can be observed even if the fifth fluorescence FL5 has a wavelength similar to the wavelength of the sixth excitation light EX6 or even if the wavelength of the fifth fluorescence FL5 is the same as the wavelength of the sixth excitation light EX6. Thus, the number of combinations of fluorescent agents that can be used is increased, and observation of the lesion C can be easily carried out.

The technical scope of the present invention is not limited to the embodiments described above, and various modifications may be made to the present invention so long as these modifications do not deviate from the technical scope of the present invention.

For example, in the above, an endoscope apparatus using excitation light in two difference spectral bands and fluorescence in two different fluorescence bands generated by the excitation light according to an embodiment was described. However, the excitation light and the fluorescence are not limited to two spectral bands: the endoscope apparatus may use excitation light and fluorescence in more than three bands.

What is claimed is:

1. An endoscope apparatus comprising:
an illumination unit configured to emit illumination light including light in a visible spectral band and excitation light in a plurality of spectral bands that generates fluorescence in a plurality of different infrared bands toward an examination region in a body to be examined;
an imaging unit configured to capture the illumination light reflected at the examination region and the fluorescence generated at the examination region, the imaging unit being disposed at a distal end of an insertion section to be inserted inside the body to be examined; and
a cut filter configured to block at least the excitation light in the plurality of spectral bands, the cut filter being disposed adjacent to the imaging unit on a light-incident side.

2. The endoscope apparatus according to claim 1, wherein the illumination unit comprises a switching unit configured to switch between the illumination light and the excitation light so that the illumination light and the excitation light are emitted in sequence.

3. The endoscope apparatus according to claim 2, wherein the illumination unit further comprises a visible light emission unit configured to emit the illumination light and an excitation light emission unit configured to emit the excitation light.

4. The endoscope apparatus according to claim 1, wherein the cut filter blocks the excitation light in the plurality of spectral bands and transmits fluorescence in at least one of the fluorescence bands, said at least one of the fluorescence bands being a spectral band located between the spectral bands of the excitation light.

5. The endoscope apparatus according to claim 1, wherein the cut filter blocks light in a range of wavelengths corresponding to a single, uninterrupted spectral band.

6. The endoscope apparatus according to claim 1, further comprising:
a fluorescence determination unit for determining whether fluorescence in a predetermined fluorescence band is generated in a predetermined region of the body to be examined on the basis of a fluorescence image captured by the imaging unit.

7. An endoscope apparatus comprising:
an illumination unit configured to emit illumination light including light in a visible spectral band and excitation light in a plurality of spectral bands that generates fluorescence in a plurality of different infrared bands toward an examination region in a body to be examined;
a first imaging unit configured to capture at least a portion of the fluorescence generated at the examination region, the first imaging unit being disposed at a distal end of an insertion section to be inserted in the body to be examined;
a second imaging unit configured to capture all or a portion of the fluorescence included in at least the illumination light reflected at the examination region and the fluorescence generated at the examination region, said all or a portion of the fluorescence being the fluorescence other than the fluorescence captured by the first imaging unit, the second imaging unit being disposed at the distal end of the insertion section to be inserted in the body to be examined;
a first cut filter configured to block said at least excitation light that generates said portion of the fluorescence, the first cut filter being disposed adjacent to the first imaging unit on a light-incident side, and
a second cut filter configured to block said all or a portion of excitation light other than the excitation light which is blocked by the first cut filter, the second cut filter being disposed adjacent to the second imaging unit on a light-incident side.

8. The endoscope apparatus according to claim 7, wherein the illumination unit comprises a switching unit configured to switch between the illumination light and the excitation light so that the illumination light and the excitation light are emitted in sequence.

9. The endoscope apparatus according to claim 8, wherein the illumination unit comprises a visible light emission unit configured to emit illumination light and an excitation light emission unit configured to emit excitation light.

10. The endoscope apparatus according to claim 7, wherein at least one of the first cut filter and the second cut filter block the excitation light in a plurality of spectral bands and transmit fluorescence in at least one of the fluorescence bands, said at least one of the fluorescence bands being a spectral band located between the spectral bands of the excitation light.

11. The endoscope apparatus according to claim 7, wherein at least one of the first cut filter and the second cut filter blocks light in a range of wavelengths corresponding to a single, uninterrupted spectral band.

12. The endoscope apparatus according to claim 7, further comprising:
a fluorescence determination unit for determining whether fluorescence in a predetermined fluorescence band is generated in a predetermined region of the body to be examined on the basis of a fluorescence image captured by the imaging unit.

13. A method for determining whether fluorescence is generated using an endoscope apparatus, comprising the steps of:
emitting excitation light in a plurality of spectral bands from an insertion section of an endoscope apparatus into a body to be examined;
blocking the excitation light reflected at the body to be examined while capturing an image corresponding to fluorescence generated inside the body to be examined, the fluorescence being in a plurality of spectral bands; and
determining whether fluorescence in a predetermined fluorescence band has been generated in a predetermined region of the body to be examined on the basis of the fluorescence images captured in the blocking step.

14. The method for determining whether fluorescence is generated using an endoscope apparatus according to claim 13, wherein the determining step comprises the steps of:
measuring the amount of fluorescence in one of the fluorescence bands among the plurality of fluorescence bands generated at the predetermined region;
determining whether fluorescent agents used to generate the fluorescence in the plurality of fluorescence bands are spread throughout the predetermined region on the basis of the amount of fluorescence; and
detecting fluorescence in another fluorescence band after the fluorescent agents are determined to be spread throughout the predetermined region in the determining step.
